(19) United States
(12) Patent Application Publication
(10) Pub. No.: US 2012/0157985 A1
(43) Pub. Date: Jun. 21, 2012

(54) GLOBAL ENDOMETRIAL ABLATION DEVICE

(76) Inventors: Lisa M. BALLOU, Santa Clara, CA (US); Melissa D. Caputo, San Jose, CA (US); Pajhand Iranitalab, San Ramon, CA (US); Xiaodong Xiang, Milpitas, CA (US)

(21) Appl. No.: 13/328,603

(22) Filed: Dec. 16, 2011

Related U.S. Application Data
(60) Provisional application No. 61/459,725, filed on Dec. 17, 2010.

(51) Int. Cl. A61B 18/18

(52) U.S. Cl. 606/33

(57) ABSTRACT

An endometrial ablation apparatus is provided which includes an end member with a plurality of electrodes. The probe is attached to a controller that includes a multiplexer capable of activating each electrode individually or multiple electrodes simultaneously, such that each individual electrode may be energized separately in series to complete the ablation process. A method of performing ablation using such an apparatus is also provided.

### CROSS REFERENCE TO RELATED APPLICATIONS

This application claims the benefit of U.S. Provisional Application No. 61/459 725, filed Dec. 17, 2010, the disclosure of which is hereby incorporated by reference in its entirety.

### BACKGROUND OF THE INVENTION

The present invention is related generally to ablation devices, and more particularly to endometrial ablation devices and methods using radio frequency energy.

Approximately 20% of women experience excessive prolonged menstrual bleeding at some point during their adult lives. As an alternative to hormone pills or hysterectomy procedures, the less invasive procedure of global endometrial ablation (“GEA”) preserves the uterus, while decreasing menstrual bleeding and allowing the patient a shorter recovery time from the procedure.

GEA destroys the endometrial lining within the uterine cavity. It involves only minimally invasive surgery, which may be outpatient in nature. The procedure involves the use of an energy source, such as heat, cold, microwave energy, and/or radio frequency energy, to destroy the endometrial lining while leaving the uterus intact.

A variety of ablation devices have been marketed and used. However, known ablation devices have shortcomings that result in less than ideal results for a GEA procedure. The shortcomings result in efficacy rates being below 40%. Moreover, known devices may result in severe adverse events, including perforation of the uterus and bowel, as well as burns. In addition, known devices cannot contour to abnormally-shaped or abnormally-sized uterine cavities, making some women ineligible for the procedure. It is also known that the applied energy used during the ablation procedure for the currently marketed devices can be inefficiently and unevenly distributed, which may result in unnecessary burn depths.

As a result, the inventors herein have developed an endometrial ablation device that is safer and more effective than currently marketed devices. One embodiment of such an endometrial ablation device includes a disposable ablation probe having multiple electrodes and a sensor for determining the impedance across each electrode. The sensor sends a signal to a controller, which calculates the impedance across a given electrode of the probe. A monopolar radio frequency (RF) generator is also included which generates and delivers monopolar radio frequency energy to the electrodes. The controller is attached to both the impedance sensor and the RF generator, so that each electrode may be separately energized based on data signals from the impedance sensor. A grounding device is also used for grounding the RF energy delivered by the RF generator. The ablation apparatus may also include a conductive gel for engagement with the electrodes within a body cavity, such as a uterine cavity, to increase the conductivity of the electrical output from the electrodes. A catheter along the length of the probe, preferably within a shaft, may be employed to deliver the conductive gel from outside the body cavity to inside the body cavity. The flexible probe may also comprise an end member that is fan-shaped and expandable within the body cavity to increase ease of insertion and efficiency of use.

In use, the preferred ablation device described above is provided with an RF controller having a multiplexer. To perform a GEA, the end member of the flexible probe is inserted into a uterine cavity of a patient. Inserting conductive gel and circulating the gel within the body cavity, to provide increased conductivity, are also preferred. A first electrode is energized by use of the controller until a predetermined impedance level is detected by the controller due to a signal from the sensor. Once the predetermined impedance level is detected, the first electrode is de-energized. A second electrode is then energized by the RF controller and remains energized until a predetermined impedance level for that electrode is reached, at which time the second electrode is de-energized. This process is repeated for as many electrodes or combinations of electrodes are needed to complete the ablation process. Alternatively, a plurality of electrodes are energized simultaneously. After the ablation is completed, the end member and gel are removed from the uterine cavity.

Certain terminology will be used in the following description for convenience in reference only, and will not be limiting. For example, the words “upwardly”, “downwardly”, “rightwardly” and “leftwardly” will refer to directions in the drawings to which reference is made. The words “inwardly” and “outwardly” will refer to directions toward and away from, respectively, the geometric center of the end member or shaft of the ablation apparatus, and designated parts thereof. Said terminology will include the words specifically mentioned, derivatives thereof, and words of similar import.

### BRIEF DESCRIPTION OF THE DRAWINGS

FIG. 1 is a longitudinal cross sectional elevational view of the female reproductive system and a first embodiment of the present endometrial ablation apparatus invention.

FIG. 2 is a side elevational view of an end member of one embodiment of a flexible probe of the endometrial ablation apparatus of the present invention, depicting one electrode in an activated state.

FIG. 3 is a cross sectional view taken substantially along line III-III in FIG. 2 of a first embodiment of a probe of the endometrial ablation device of FIG. 2.

FIG. 4 is a longitudinal cross sectional elevational view of the female reproductive system and a second embodiment of the present endometrial ablation apparatus invention.

FIG. 5A is a transverse cross sectional view, taken substantially along line V-V in FIG. 4, of an embodiment of a probe having a flow lumen and a fanned tip insert.

FIG. 5B is a transverse cross sectional view, taken substantially along line V-V in FIG. 4, of another embodiment of a probe having flow lumens and a fanned tip insert.

FIG. 5C is a transverse cross sectional view, taken substantially along line V-V in FIG. 4, of yet another embodiment of a probe having a flow lumen and a fanned tip insert.

FIG. 5D is a transverse cross sectional view, taken substantially along line V-V in FIG. 4, of still another embodiment of a probe having flow lumens and a fanned tip insert.

FIG. 6 is a flow chart showing preferred process steps of endometrial ablation using the endometrial ablation apparatus of the present invention.

### DETAILED DESCRIPTION OF THE PREFERRED EMBODIMENTS

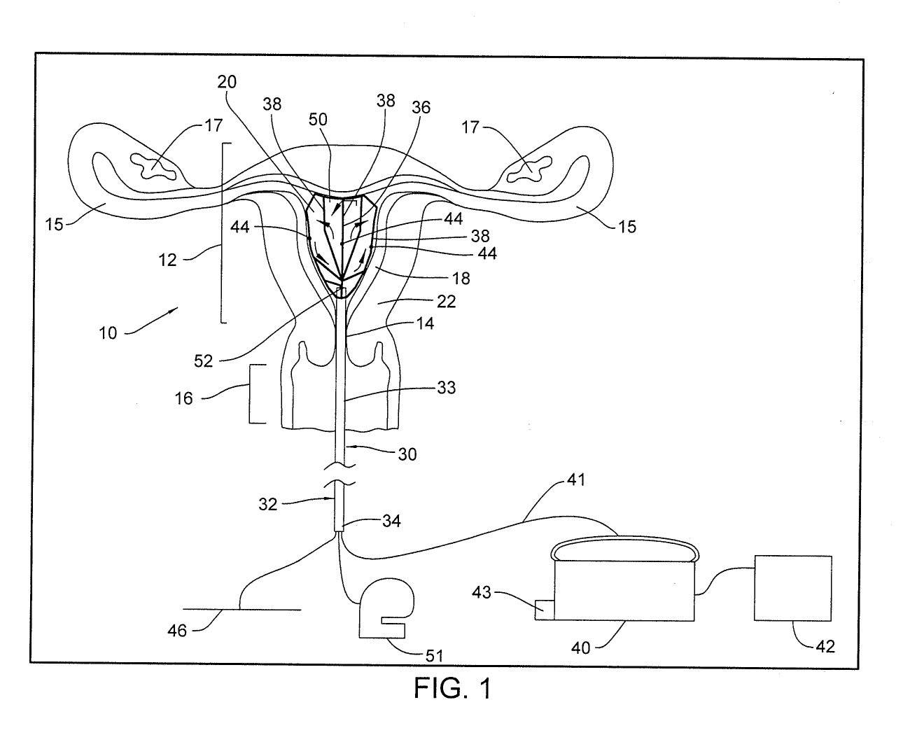

The ablation device embodiments of the present invention are for use in a body cavity, such as in the uterine cavity of the female reproductive system 10, shown in FIG. 1. The female reproductive system 10 includes, among other things, a uterus 12 which includes a cervix 14, fallopian tubes 15, a vagina 16, and ovaries 17. The uterus has a thick lining layer, endometrium 18, that defines a uterine cavity 20 and a muscular wall, myometrium 22.

An endometrial ablation device 30 generally includes a probe 32, preferably disposable, which has a tubular or hollow shaft 33, a handpiece 34 attached to or part of the shaft 33 at its proximal end, and an end member 36 at the distal end of the shaft 33. End member 36 is comprised of a number of electrodes 38. Wires or other electrical conductive material or media extends between the electrodes 38 and the proximal end of the probe 32. The proximal end of the probe 32 is connected to a controller 40 via a cable 41. The controller 40 includes, or is alternately attached to, a monopolar radio frequency generator 42. The controller 40 preferably includes a multiplexer 43 such that the controller has the ability to enable and disable electrodes 38 individually and separately from each other. Optionally, the controller 40 can be capable of controlling current density by having the ability to energize a plurality of electrodes 38 simultaneously. The end member 36 includes one or more sensors 44, located on or adjacent electrodes 38 which detect current through a given electrode 38 and voltage across that given electrode 38. In turn, this data is sent to the RF controller 40 and is used to determine the impedance at a given time across a given electrode 38. The RF controller 40 is programmed such that once a predetermined impedance level is reached, the given electrode 38 is then disabled (i.e. de-energized) and the next or following electrode enabled. The electrodes 38 are monopolar, and thus a grounding pad 46 is also preferably included to control the current path. The grounding pad 46 may be placed on the patient perpendicular to the desired current path so that the desired current path will follow the current vector normal to the desired endometrial region of the uterine cavity that is being ablated.

Preferably, a conductive gel 50 is inserted into the uterine cavity 20 prior to the ablation process to increase the efficiency and balance the electrical current during ablation. The conductive gel 50 is of higher electrical conductivity than the electrical conductivity of the tissue of the endometrium 18 to maximize the energy transfer to the tissue by decreasing resistance between the energized electrode 38 and adjacent tissue and by bridging the gaps between the electrodes 38 and tissue that are not directly in contact. The use of the conductive gel 50 allows more energy to be delivered to the tissue resulting in faster ablation times. The conductive gel 50 is preferably a viscous substance, thus discouraging the gel from penetrating through perforations and decreasing the potential for adverse effects. The conductive gel 50 is pumped to the distal end of the ablation probe 32 through shaft 33 and into a body cavity by use of a gel pump 51. The conductive gel 50 can be either stationary during the ablation process or be circulated during ablation as indicated by the arrows in FIG. 1.

The probe 32 also preferably includes a pressure sensor 52 for monitoring the pressure of the conductive gel 50 in the uterine cavity 20. Pressure data signals are relayed to the RF controller 40 and any significant decrease in pressure of the conductive gel 50 indicates a potential perforation or leak into the cervix or the fallopian tubes. Therefore, monitoring the pressure of the conductive gel 50 in the uterine cavity 20 acts as a safety test during the ablation process.

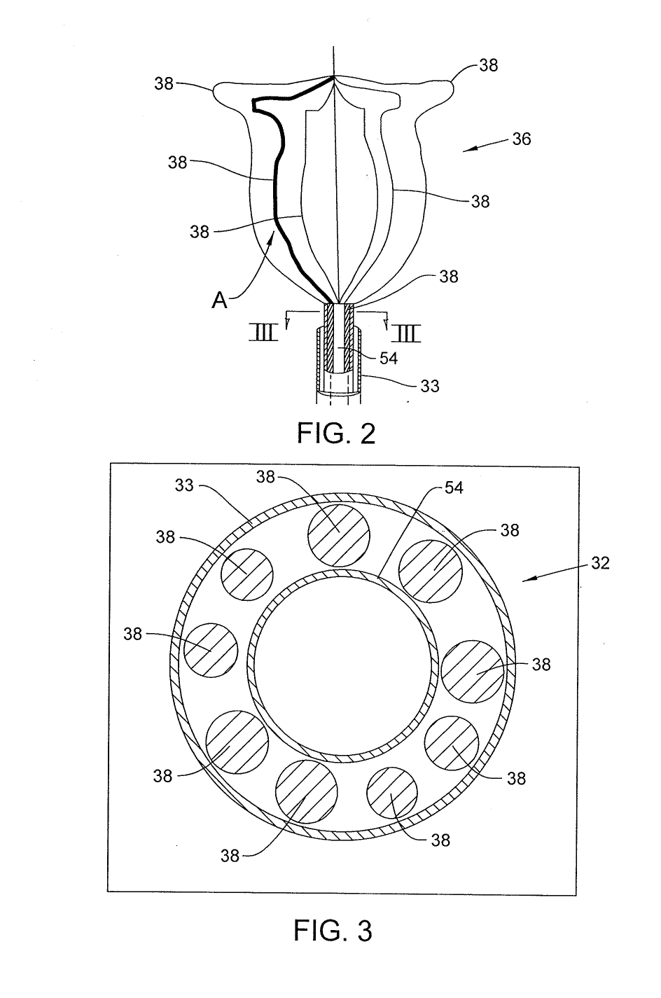

As shown in FIG. 2, end member 36 includes a plurality of electrodes 38 which in the illustrated embodiment are preferably circumferentially spaced apart substantially evenly from each other which provides end member 36 with a cage-like configuration. Preferably, each electrode 38 can be individually and separately energized and de-energized. A single energized electrode 38 is denoted by the letter A in FIG. 2.

FIG. 3 shows a preferred substantially evenly-spaced relationship of the electrode wires 38 through the probe 32. A catheter 54, which is preferably centrally located in probe 32, is also depicted. The catheter 54 delivers conductive gel 50 from outside the body cavity, such as the uterine cavity 20, into the body cavity prior to energizing of one or more of the electrodes 38.

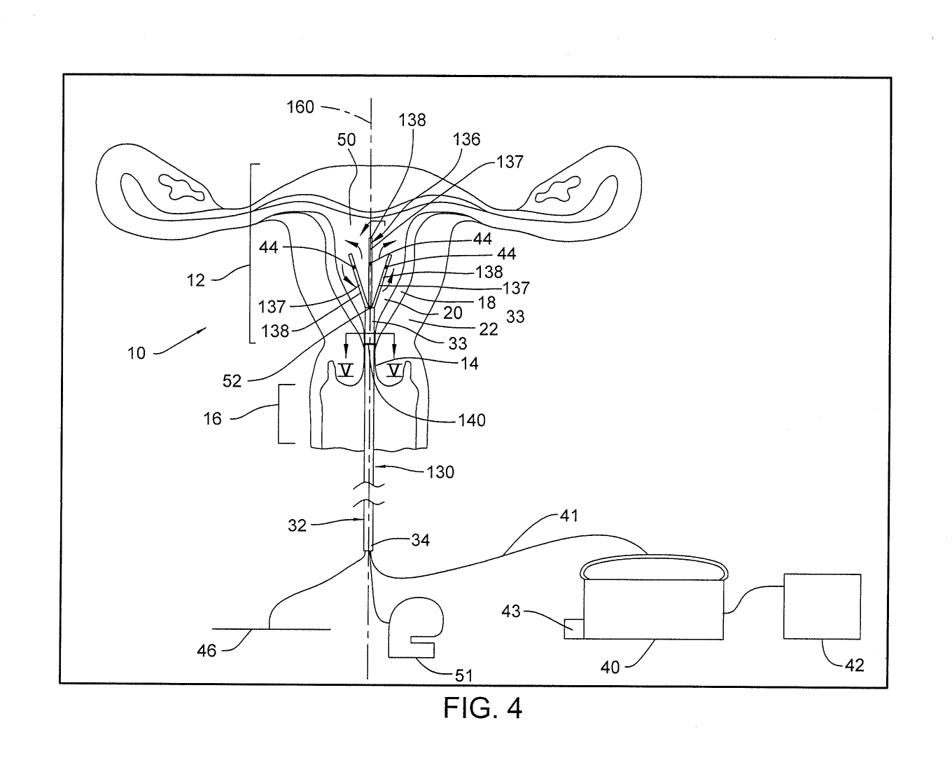

In a second embodiment shown in FIG. 4, an endometrial ablation device 130 has all of the same components as the device described above with the exception of the end member. In the second embodiment, the end member is designated as part 136, shown in FIG. 4. End member 136 includes a plurality of fins 137, each of which includes an electrode 138. Fins 137 are arranged or arrangeable in a fan-like configuration, that is the end member 136 is radially or outwardly expandable and retractable with respect to longitudinal axis 160 so that insertion of the end member 136 into the uterine cavity 20 is easier. After insertion into the uterine cavity 20, end member 136 can be expanded outwardly such that the electrodes 138 are positioned adjacent the tissue of the endometrium 18. As with the first embodiment, a conductive gel 50 is preferably used for enhanced conductivity of the energy that the electrodes 138 radiate. The conductive gel 50 may be stagnant or circulated. Optionally, a cervical plug 140 may be used to eliminate, or at least minimize, leakage of conductive gel 50 from the uterine cavity 20.

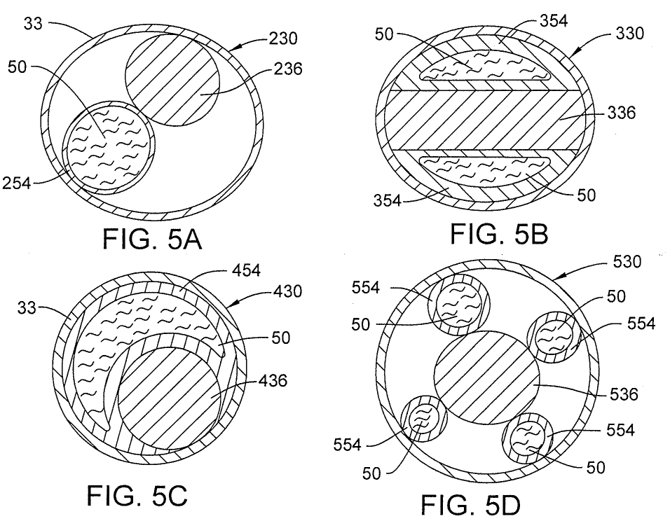

FIGS. 5A-5D show four cross sections of probe 130. The cross sections show various constructions of the probe 130 with the fanned tip end member 136 and the catheter 54. FIG. 5A depicts probe 230 having an end member 236 and a tubular catheter 254 that are roughly the same size in cross-sectional dimension and positioned side-by-side in probe 230. Catheter 254 carries gel 50 from the proximal end to the distal end of probe 230. FIG. 5B shows probe 330 having therein a generally rectangular-shape (in cross section) fanned tip 336 and a catheter 354. Catheter 354, which delivers gel 50 from the proximal end of probe 330 to its distal end, is divided into two parts, which substantially encompass the volume within probe 330 not taken up by tip 336. FIG. 5C shows a probe 430 having a generally circular-shaped (in cross section) fanned tip end member 436 and a catheter 454 which encompasses the inner volume of probe 430 that end member 436 does not take up. Catheter 454 defines a passageway to deliver gel 50 from the proximal end to the distal end of probe 430. FIG. 5D depicts probe 530, which has a generally centrally-positioned fanned tip 536 therein and multiple catheters 554, which are each smaller in diameter in cross-sectional dimension than the fanned tip 536. Each catheter 554 is capable of transporting gel 50 from the proximal end of probe 530 to the distal end of probe 530.

In operation, a disposable probe having the structure of one of the embodiments above 30, 130, 230, 330, 430, 530 is attached to a controller 40 with a multiplexer 43, which in turn is attached to, or includes, monopolar radio frequency generator 42. The probe is also attached to grounding pad 46 for control of the current. The end member 36, 136, 236, 336, 436, 536 at the distal end of the shaft 33 of the flexible probe is inserted into a female patient through the vagina and into the uterine cavity 20. If an embodiment with the fanned tip end member 136, 236, 336, 436, 536 is being employed, the fanned tip end member is then expanded to move the electrodes 138 adjacent the tissue of the endometrium 18. After insertion of the end member, conductive gel 50 is preferably dispersed into the uterine cavity through catheter 54, 254, 354, 454, 554 (and optionally cervical plug 140 may be employed). Pressure sensor 52 detects the pressure within the uterine cavity 20, and relays the signal back to the controller 40, which controls the flow of conductive gel 50 into the uterine cavity 20. Once a predetermined pressure level is detected by the controller 40, insertion of the conductive gel 50 is stopped. If circulation of the conductive gel 50 is desired, at this point circulation is initiated, after which one of the electrodes 38, 138 of the end member 36, 136, 236, 336, 436, 536 is activated.

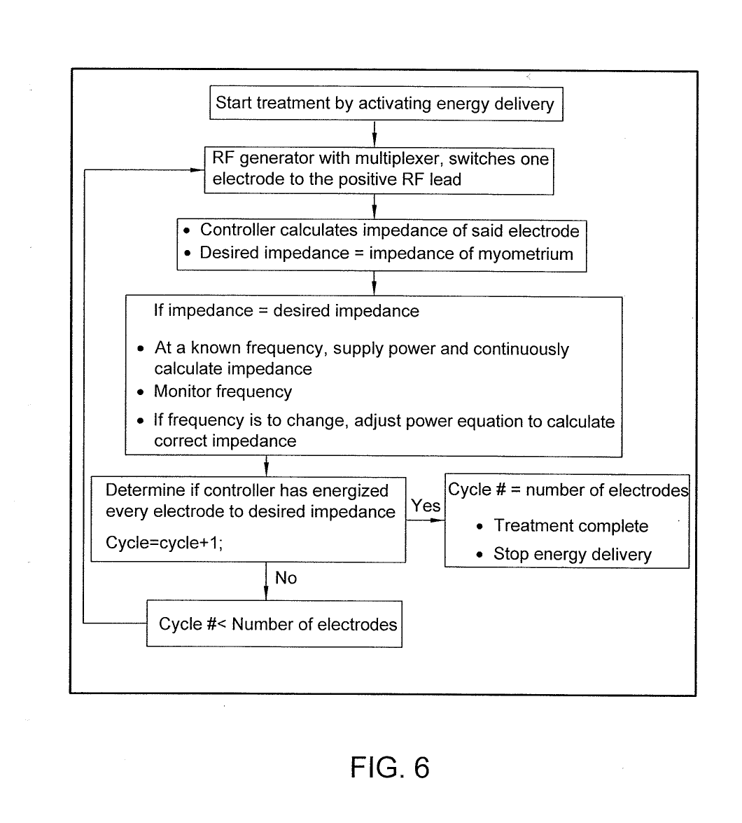

FIG. 6 shows the preferred procedure/algorithm to accomplish the ablation using one of the above-described ablation apparatus embodiments. The treatment is started by activating energy delivery to the controller 40. The RF generator 42 and controller 40, which includes multiplexer 43, connects one electrode to the positive RF lead thereby energizing that electrode to initiate the ablation process. The impedance sensors 44 detect impedance levels and send one or more data signals to the controller 40. The preferred impedance is typically the impedance of the myometrium 22. The controller 40 calculates the impedance. Once the desired impedance is reached for a particular electrode, that electrode is deactivated, and the following electrode in series is activated until the desired impedance for that electrode is reached. The controller 40 determines if each electrode has been energized to the desired impedance. If not, the operation continues to activate the following electrode in series, until all of the electrodes have been energized and have reached the desired impedance. Once the electrodes have all been energized and have reached the desired impedance, the treatment is complete and energy delivery is stopped.

Alternatively, multiple electrodes may be activated simultaneously. If less than all of the electrodes are energized at one time, once one set of electrodes completes the ablation process, a new set of electrodes is energized, and the process repeated until all electrodes have completed the ablation process.

The above described apparatus and method of ablation result in a safer and more effective and efficient ablation procedure and device as contrasted with currently marketed devices. The inventive apparatus is easy to use and provides safe ablation with minimized risk of perforations or burns.

Although particular preferred embodiments of the invention have been disclosed in detail for illustrative purposes, it will be recognized that variations or modifications of the disclosed apparatus, including the rearrangement of parts, lie within the scope of the present invention.

What is claimed is:

1. An ablation apparatus comprising:
an ablation probe having a plurality of electrodes for generating electrical energy output and a sensor for determining the impedance separately across each electrode, the sensor disposed to send a data signal regarding the impedance across a given electrode;
a monopolar radio frequency generator disposed to generate and deliver radio frequency energy;
a controller connected to the impedance sensor and connected to the monopolar radio frequency generator, and configured to receive data signals from the sensor for each of the electrodes of the ablation probe and to selectively and separately energize each of the electrodes to a radio frequency energy level, the radio frequency controller being configured to reduce, increase, or maintain the radio frequency energy level for an electrode based on a data signal received from the sensor relative to a given electrode; and
a grounding device connected to the disposable ablation probe for grounding the monopolar radio frequency energy delivered by the radio frequency generator.

2. The ablation apparatus of claim 1, wherein the controller has an integrated multiplexer.

3. The ablation apparatus of claim 1, wherein the controller is configured to continuously calculate the impedance of an electrode by the measurement of current through the electrode and voltage across the electrode.

4. The ablation apparatus of claim 1, wherein the controller is configured to control current density by energizing a plurality of electrodes simultaneously.

5. The ablation apparatus of claim 1, and further comprising a conductive gel for engagement with the electrodes to increase the conductivity of the electrical output from the electrodes during an ablation procedure.

6. The ablation apparatus of claim 5, and further comprising a gel pump for pumping the conductive gel, and a pressure sensor capable of sensing the pressure of conductive gel in a uterine cavity during an ablation procedure.

7. The ablation apparatus of claim 6, wherein the ablation probe further comprises a catheter which is attachable to the gel pump for distribution of conductive gel into a body cavity during an ablation procedure.

8. The ablation apparatus of claim 5, wherein the conductive gel has an electrical conductivity greater than the electrical conductivity of uterine tissue.

9. An ablation apparatus comprising:
a flexible probe comprising a proximal end and a distal end, an end member at the distal end, the end member comprising a plurality of electrodes, each electrode for receiving monopolar radio frequency signals from a controller and a handpiece at the proximal end, each electrode capable of generating electrical output when energized by the controller; and
an expanding member attached to and extending between the end member and the handpiece,
the end member being fan-shaped and expandable in a uterine cavity by movement of the expanding member.

10. The ablation apparatus of claim 9, the flexible probe further comprising fins to which the electrodes are attached.

11. The ablation apparatus of claim 9, and further comprising an elongated shaft to which the end member is attached and to which the handpiece is attached.

12. The ablation apparatus of claim 11, the elongated shaft having an interior, and the ablation apparatus further comprising a catheter positioned in the interior of the elongated shaft, the catheter disposed to deliver gel to the distal end of the flexible probe.

13. The ablation device of claim 12, and further comprising a cervical plug for prevention of gel leakage from a uterine cavity.

14. A method of ablating an endometrium, comprising the steps of:
(a) providing an ablation probe having an end member comprising a first electrode and a second electrode, and an impedance sensor for sensing the impedance separately across an electrode;
(b) providing a controller having a multiplexer, the controller connected to the impedance sensor and to the first electrode and second electrode;
(c) providing a monopolar radio frequency generator attached to and controlled by the controller;
(d) inserting the end member into a body cavity;
(e) energizing the first electrode by use of the controller until a predetermined impedance level is reached, at which time the first electrode is de-energized by the controller;
(f) energizing the second electrode by use of the controller until a predetermined impedance level is reached, at which time the second electrode is de-energized; and
(g) removing the end member from the body cavity.

15. The method of claim 14, and further including the step of inserting a conductive gel into the body cavity prior to the step of energizing the first electrode.

16. The method of claim 15, and further including the step of circulating the conductive gel within the body cavity after the step of inserting the conductive gel into the body cavity.

17. The method of claim 14, wherein the end member is fan-shaped, and is expanded after insertion into a body cavity.

18. The method of claim 15, wherein the ablation probe comprises a pressure sensor and the method further includes the step of using the pressure sensor to detect the pressure of the conductive gel in the body cavity prior to the step of energizing the first electrode.
